(19) United States
(12) Patent Application Publication
(10) Pub. No.: US 2017/0181664 A1
(43) Pub. Date: Jun. 29, 2017

(54) SEDATION SYSTEM WITH MULTI-INPUT CAPNOMETER

(71) Applicant: Ethicon Endo-Surgery, Inc., Cincinnati, OH (US)

(72) Inventors: Paul J. Niklewski, Cincinnati, OH (US); James F. Martin, Lebanon, OH (US)

(21) Appl. No.: 14/982,172

(22) Filed: Dec. 29, 2015

(51) Int. Cl. A61B 5/083; A61M 16/08; A61M 16/20; A61M 16/01; A61M 16/06

(52) U.S. Cl. CPC A61B 5/0836; A61M 16/01; A61M 16/0666; A61M 16/202; A61M 16/208; A61M 16/085; A61M 2205/3303; A61M 2205/50; A61M 2230/432

(57) ABSTRACT

An oral-nasal cannula receives exhaled gases from the nose and mouth of a patient. The exhaled gases are transported to variable flow valves that can variably restrict the flow of the gases through the valves upon software generated signals. The exhaled gases pass through the variable flow valves and mix so that they can be measured by a single sensor such as a sensor of a capnometer. Based upon information gathered by the capnometer, the variable valves can be adjusted in real-time according to a software method in order to identify a variable valve flow configuration that maximizes the amount of CO₂ received and measured by the capnometer. In this manner, the software can adapt a single capnometer to measure exhaled gases regardless of whether a patient breathes primarily through their nose or mouth or some proportion of the two.

### BACKGROUND

Patient monitoring systems may be used to monitor physiological parameters of patients undergoing diagnostic procedures, surgical procedures, and/or various other types of medical procedures. In various settings, it may also be desirable to deliver drugs to a patient during a procedure, such as via an IV and/or face mask, etc. Such drugs may include sedatives, anelgesics, amnestics, etc. In some instances, such drugs may be selected and/or combined to place a patient in a state of “conscious sedation” (in lieu of simply rendering a patient completely unconscious through a general anesthetic). Certain systems may also be used to automate the delivery of such drugs. For instance, such systems may be located in the same room where a medical procedure is performed, and may be coupled with a physiological monitoring system to automatically tailor the delivery of drugs based on patient parameters detected by the monitoring system.

Examples of such systems are disclosed in U.S. Pat. No. 6,745,764, entitled “Apparatus and Method for Providing a Conscious Patient Relief from Pain and Anxiety Associated with Medical or Surgical Procedures,” issued Jun. 8, 2004, the disclosure of which is incorporated by reference herein; U.S. Pat. No. 7,833,213, entitled “Patient Monitoring and Drug Delivery System and Method,” issued Nov. 16, 2010, the disclosure of which is incorporated by reference herein; U.S. Pat. No. 7,935,081, entitled “Drug Delivery Cassette and a Medical Effector System,” issued May 3, 2011, the disclosure of which is incorporated by reference herein; U.S. Pub. No. 2009/0292179, entitled “Medical System having a Medical Unit and a Display Monitor,” published Nov. 26, 2009, the disclosure of which is incorporated by reference herein; and U.S. Pub. No. 2010/0010433, entitled “Medical System which Controls Delivery of a Drug,” published Jan. 14, 2010, the disclosure of which is incorporated by reference herein.

Some such monitoring system measure the respiratory gases produced by a patient undergoing a procedure via, for example, an oral/nasal cannula such as that disclosed in U.S. Pat. No. 7,935,081, referenced above. One difficulty in measuring respiratory gases exhaled by a patient is that exhalation may occur through both oral and nasal passageways, and the reliance on the oral passageway versus the nasal passageway may vary greatly from one patient to another. For example, a first patient may primarily breathe through their nose when at rest, whereas another patient may primarily breathe through their mouth when at rest. To measure gases exhaled from both the nose and mouth in order to account for varying breathing habits, some oral/nasal cannulas may separately capture the gases exhaled from the nose and mouth so that they may be isolated and measured by separate sensors. However, this solution requires duplication and redundancy of components which may increase the complexity and expense of a resulting device. A more complex and expensive device may, depending upon a particular implementation and purpose, be disadvantageous.

While a variety of systems have been made and used for measuring exhaled respiratory gases from a patient, it is believed that no one prior to the inventor(s) has made or used the technology as described herein.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1 depicts a perspective view of an exemplary patient monitoring and drug delivery system;

FIG. 2 depicts a perspective view of the patient monitoring unit of the system of FIG. 1;

FIG. 3 depicts a perspective view of the drug delivery unit of the system of FIG. 1;

FIG. 4 depicts a block diagrammatic view of the system of FIG. 1 with additional exemplary components;

FIG. 5 is a perspective view of an oral/nasal cannula that may be used with the system of FIG. 1, with connecting tubes omitted for clarity;

FIG. 6 is a schematic view of an embodiment of an exemplary capnometer system that may be used with the system of FIG. 1;

FIG. 7 depicts a flowchart of exemplary steps that may be performed using the capnometer system of FIG. 6 to manage a variable valve input to a capnometer of the capnometer system;

FIG. 8 depicts a flowchart of exemplary steps that may be performed using the capnometer system of FIG. 6 to pre-configure a set of variable valves of the capnometer system;

FIG. 9 depicts a flowchart of exemplary steps that may be performed using the capnometer system of FIG. 6 to pre-configure a set of variable valves of the capnometer system using a sweep method;

FIG. 10 depicts a flowchart of exemplary steps that may be performed using the capnometer system of FIG. 6 to receive exhaled gases via a variable valve input of the capnometer system; and

FIG. 11 depicts a flowchart of exemplary steps that may be performed using the capnometer system of FIG. 6 to adjust a set of variable valves of the capnometer system in response to a previous input.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview

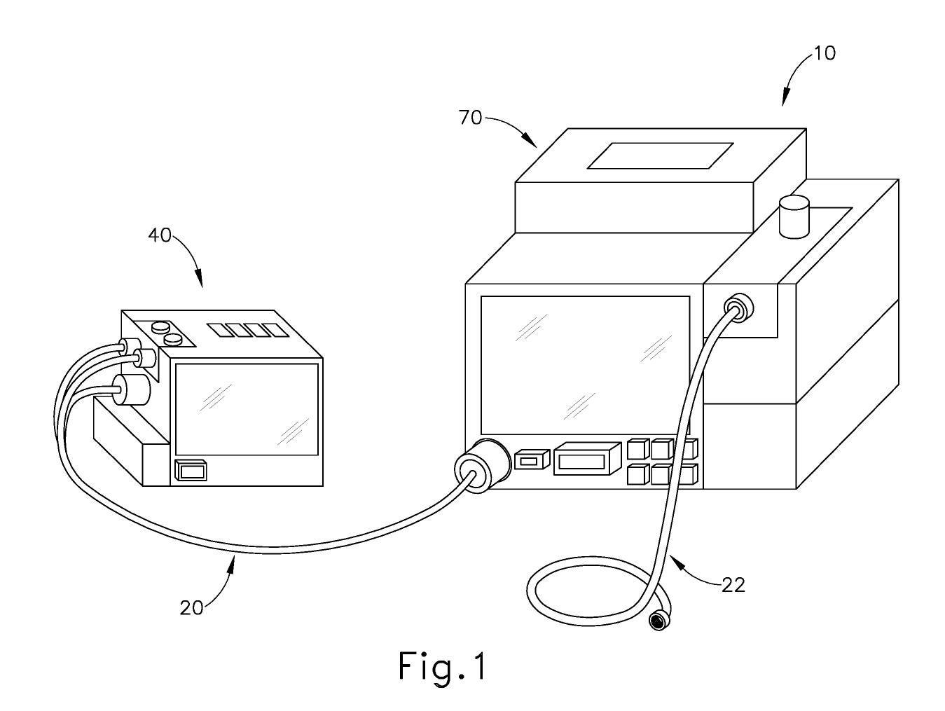

FIG. 1 shows an exemplary patient care system (10) comprising a bedside monitor unit (BMU) (40) and a procedure room unit (PRU) (70). One exemplary use of patient care system (10) is to monitor patient parameters and deliver sedative, analgesic, and/or amnestic drugs to a conscious, non-intubated, spontaneously-ventilating patient undergoing a diagnostic procedure, surgical procedure, or other medical procedure by a physician. This use is not exhaustive of all of the potential uses of the invention but will be used to describe examples herein. BMU (40) and PRU (70) are connected via communication cable (20). Communication cable (20) provides means for transmitting electronic data as well as various hydraulic signals and gases between BMU (40) and PRU (70). For instance, communication cable (20) may include a plurality of pneumatic tubes and a plurality of electrical wires, all integrated within a single sheath or cable. Communication cable (20) may be removed from both BMU (40) and PRU (70) to facilitate practice efficiency and user convenience. BMU (40) and PRU (70) are free to move independently of each other if communication cable (20) is not in place. This allows for mobility of each unit independent of the other; this feature is especially important in hospitals that have a great deal of medical procedures and there is little time to connect patients to monitors. BMU (40) and PRU (70) preferably accommodate an external oxygen source that is intended to provide supplemental oxygen to the patient during the course of a surgical procedure if the clinician so desires. An IV tube set (22) is shown connected to PRU (70) and delivers sedative or amnestic drugs to a patient during a surgical procedure.

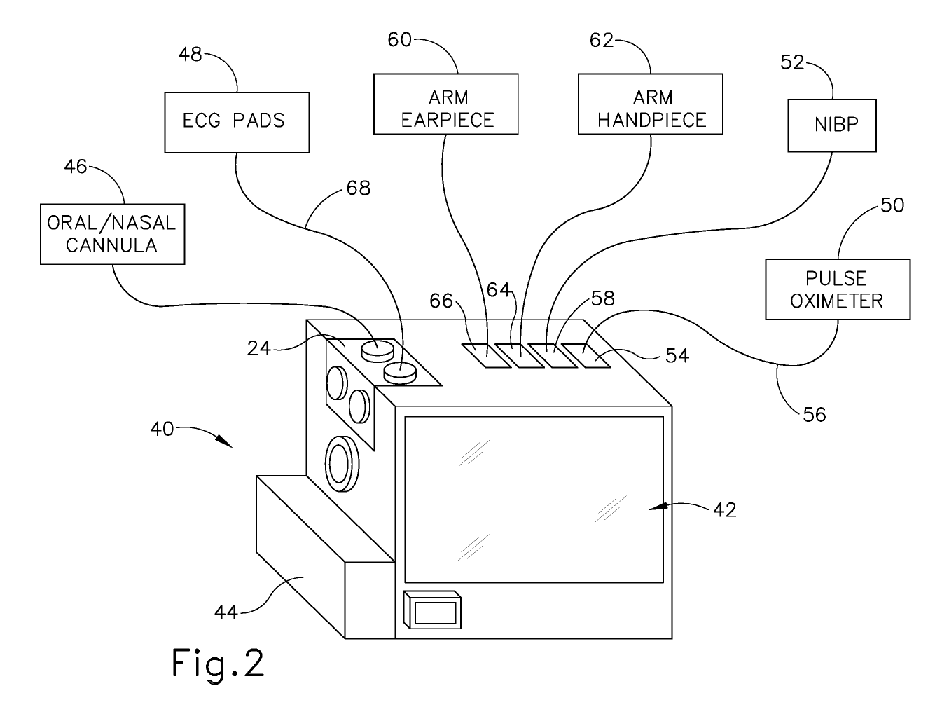

BMU (40) serves as a patient monitoring unit, monitoring various physiological parameters of a patient. As shown in FIG. 2, BMU (40) is compact and portable so it requires relatively little effort to move from one room to another. In some versions, BMU (40) could mount upon either an IV pole or a bedrail; this would free the clinician from the burden of carrying the unit wherever the patient needs to be transported. BMU (40) is small and light enough to be held in the hand of a nurse or technician. BMU (40) allows the user to input information via a touch screen assembly (42) or a simple keypad, etc. Touch screen assembly (42) is provided as an overlay on a display device that is integrated into one surface of BMU (40), and that displays patient and system parameters, and operational status of BMU (40). An exemplary bedside touch screen assembly (42) is a 5.25″ resistive touch screen manufactured by MicroTech mounted upon a 5.25″ color LCD screen manufactured by Samsung. Other suitable forms that a display screen and touch screen may take will be apparent to those of ordinary skill in the art in view of the teachings herein. An attending nurse or physician may enter patient information such as, for example, patient weight and a drug dose profile into BMU (40) by means of bedside touch screen assembly (42). A BMU battery (44) is fixedly attached to the BMU (40) and comprises a standard rechargeable battery such as, for example, Panasonic model no. LC-T122PU, that is capable of supplying sufficient power to run BMU (40) for an extended period of time. In some versions, BMU battery (44) can be recharged while BMU (40) is connected to PRU (70) via communication cable (20) or can be charged directly from an independent power source. Various suitable ways in which battery (44) may be charged will be apparent to one of ordinary skill in the art in view of the teachings herein. Similarly, various suitable forms that battery (44) may take, as well as various suitable compositions thereof, will be apparent to those of ordinary skill in the art in view of the teachings herein.

As shown in FIG. 2, BMU (40) may be connected to a plurality of patient sensors and peripherals used to monitor patient vital signs and deliver supplemental oxygen to the patient. Oral-nasal cannula (46) delivers oxygen from an external oxygen source and collects samples of exhaled gas. Oral-nasal cannula (46) is removably attached to cable pass-through connection (24). Cable pass-through connection (24) sends the signal obtained by oral-nasal cannula (46) directly to a capnometer (e.g., a CardioPulmonary Technologies CO2WFA OEM) in PRU (70) and preferably via communication cable (20) (FIG. 1). The capnometer measures the carbon dioxide levels in a patient's inhalation/exhalation stream via a carbon dioxide-sensor as well as measuring respiration rate. Also attached to the cable pass-through connection (24) is a standard electrocardiogram (ECG) (48), which monitors the electrical activity in a patient's cardiac cycle. The ECG signals are sent to the PRU (70) where the signals are processed. A pulse oximeter probe (50) (e.g., by Dolphin Medical) and a non-invasive blood pressure (NIBP) cuff (52) are also connected to BMU (40) in the present example. Pulse oximeter probe (50) measures a patient's arterial saturation and heart rate via an infrared diffusion sensor. The data retrieved by pulse oximeter probe (50) is relayed to pulse oximeter module (54) (e.g., by Dolphin Medical) by means of pulse oximeter cable (56). The NIBP cuff (52) (e.g., a SunTech Medical Instruments PN 92-0011-00) measures a patient's systolic, diastolic, and mean arterial blood pressure by means of an inflatable cuff and air pump (e.g., by SunTech Medical), also incorporated as needed. NIBP cuff (52) is removably attached to NIBP module (58) located on BMU (40).

In the present example, a patient's level of consciousness is detected by means of an Automated Responsiveness Monitor System (ARM), though like various other components described herein, an ARM system is merely optional and is not required. An exemplary ARM system is disclosed in U.S. Pub. No. 2005/0070823, entitled “Response Testing for Conscious Sedation Involving Hand Grip Dynamics,” published Mar. 31, 2005, the disclosure of which is incorporated by reference herein. The ARM system of the present example comprises a query initiate device and a query response device. The ARM system operates by obtaining the patient's attention with the query initiate device and commanding the patient to activate the query response device. The query initiate device may comprise any type of stimulus device such as a speaker via an earpiece (60), which provides an auditory command to a patient to activate the query response device. The query response device of the present example comprises is a handpiece (62) that can take the form of, for example, a toggle or rocker switch or a depressible button or other moveable member hand held or otherwise accessible to the patient so that the member can be moved or depressed by the patient upon the patient's receiving of the auditory signal or other instruction to respond. Alternatively, a vibrating mechanism may be incorporated into handpiece (62) that cues the patient to activate the query response device. For instance, in some versions, the query initiate device comprises a cylindrical handheld device (62), containing a small 12V DC bi-directional motor enabling the handheld device to vibrate the patient's hand to solicit a response.

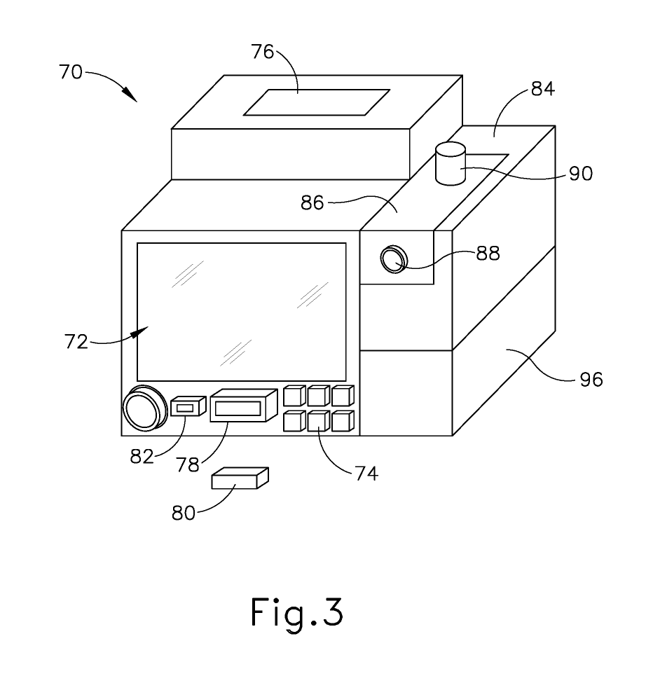

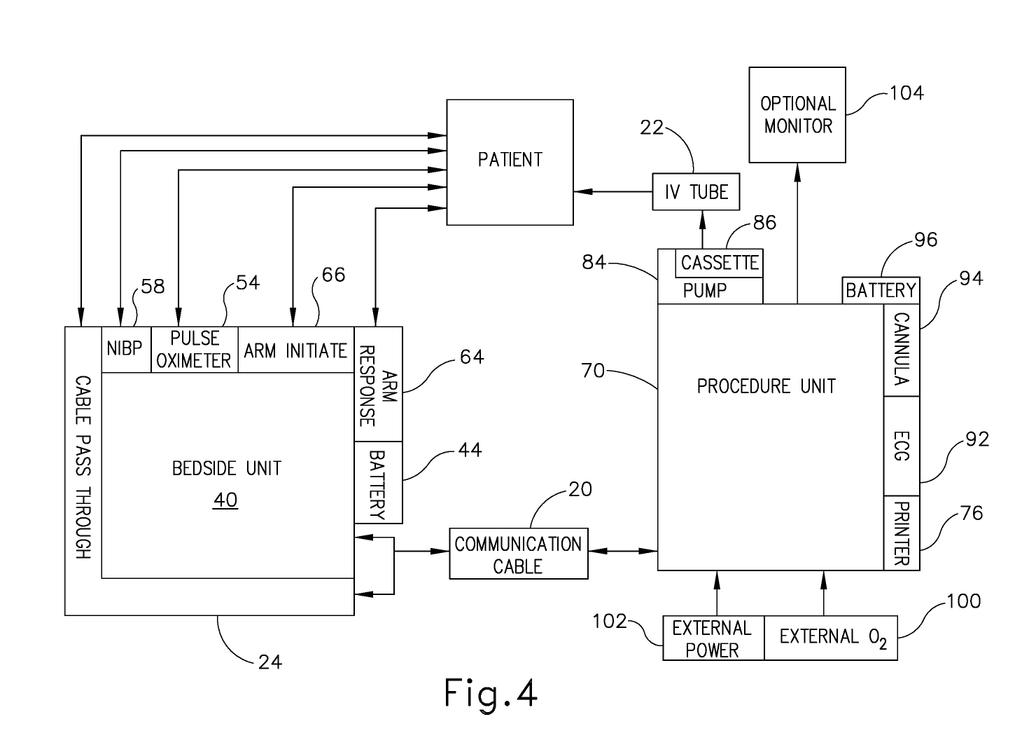

After the query is initiated, the ARM system generates signals to reflect the amount of time it took for the patient to activate the query response device in response to the query initiate device. These signals are processed by a logic board located inside BMU (40) and are displayed upon either bedside touch screen assembly (42), procedure touch screen assembly (72) (FIG. 3), and/or an optional monitor 104 (FIG. 4). The amount of time needed for the patient to respond to the query gives the clinician an idea as to the sedation level of the patient. The ARM system has two modules in this example, including a query response module (64) and a query initiate module (66), collectively referred to as the ARM system modules (64, 66). ARM system modules (64, 66) have all the necessary hardware to operate and connect the query response device (62) and the query initiate device (60) to BMU (40).

In some versions monitoring modules (54, 58, 64, 66) are easily replaceable with other monitoring modules in the event of malfunction or technological advancement. These modules (54, 58, 64, 66) include all of the necessary hardware to operate their respective peripherals. The above-mentioned patient modules (54, 58, 64, 66) are connected to a microprocessor-based electronic controller or computer (MLB) located within each of the PRU (70) and BMU (40). The electronic controller or main logic board comprises a combination of available programmable-type microprocessors and other “chips,” memory devices and logic devices on various board(s) such as, for example, those manufactured by Texas Instruments (e.g., XK21E) and National Semiconductor (e.g., HKL72), among others. Various other suitable forms that modules (54, 58, 64, 66) and associated electronics may take will be apparent to those of ordinary skill in the art in view of the teachings herein.

Once BMU (40) and PRU (70) are connected via communication cable (20), ECG and capnography may be monitored, and supplemental oxygen may be delivered to the patient. It should be understood, however, that these connections may be made in the pre-procedure room to increase practice efficiency. By making these connections in the pre-procedure room, less time may be required in the procedure room connecting capnography, ECG and supplemental oxygen to PRU (70). Oral-nasal cannula (46) and ECG leads (68) are connected directly to cable pass-through connection (24). Cable pass-through connection (24), located on BMU (40), is essentially an extension of communication cable (20), which allows the signals from ECG leads (68) and oral-nasal cannula (46) to bypass BMU (40) and be transferred directly to PRU (70). It will be evident to those skilled in the art, however, that the BMU (40) could be configured to accept the ECG (48) and oral/nasal cannula (46) signals and process the signals accordingly to provide the information on screen (42) and supplemental oxygen to the patient in the pre-procedure room. Other examples of components, features, and functionality that may be incorporated into BMU (40) will be described in greater detail below; while still further examples of components, features, and functionality that may be incorporated into BMU (40) will be apparent to those of ordinary skill in the art in view of the teachings herein.

Referring now to FIG. 3, PRU (70) allows a physician to safely deliver drugs, such as sedative, analgesic, and/or amnestic drugs to a patient, and monitor the patient during a medical procedure. Procedure touch screen assembly (72) comprises a display device that is integrated into the surface of PRU (70), which displays patient and system parameters, and operation status of PRU (70). In some versions, procedure touch screen assembly (72) comprises a 15″ resistive touch screen manufactured by MicroTech mounted upon a 15″ color LCD screen manufactured by Samsung. Other suitable forms that a display screen and touch screen may take will be apparent to those of ordinary skill in the art in view of the teachings herein. It should be noted that, in the present example, procedure touch screen assembly (72) is the primary display and user input means, and is significantly larger than the bedside touch screen assembly (42) and is capable of displaying more detailed information. In addition to procedure touch screen assembly (72), the user may input information into PRU (70) by means of drug delivery controls (74). Drug delivery controls (74), such as buttons, dials, etc., are located on one side of PRU (70) and allow the clinician to change various system parameters and bypass procedure touch screen assembly (72). A printer (76) is integrally attached to the top of PRU (70). Printer (76) allows the clinician to print a patient report that includes patient data for pre-op and the procedure itself. The combination of printing a patient report and the automatic data logging features may decrease the amount of time and effort a nurse or technician must spend regarding patient condition during the course of a procedure. Printer (76) receives data signals from a printer interface (e.g., Parallel Systems CK205HS), which is located on the main logic board. Printer (76) may comprise a thermal printer (e.g., Advanced Printing Systems (APS) ELM 205HS) and/or any other suitable type of printer. It should also be understood that printer (76) may be remote from PRU (70) and may even be omitted altogether, if desired.

Memory card reader (78), which includes a slot in the outer casing of PRU (70), allows flash memory card (80) to be inserted and removed from PRU (70). Flash memory card (80) is a solid-state storage device used for easy and fast information storage of the data log generated by PRU (70). The data is stored so that it may be retrieved from flash memory card (80) at a later time. In some versions, memory card reader (78) accepts flash memory card (80) containing software to upgrade the functionality of patient care system (10). Again, as with other components described herein, memory card reader (78) may be modified, substituted, supplemented, or omitted as desired. In the present example, memory card reader (78) is supplemented with a data port (82). Data port (82) may include, but is not limited to, a standard serial port, a USB port, a RS232 port, an Ethernet port, or a wireless adapter (e.g., using IEEE 802.11n/g/b/a standard, etc.). Data port (82) may be used to link PRU (70) to an external printer to print a patient report or to transfer electronic files to a personal computer or mainframe. Examples of how data port (82) may be used to communicate with a centralized network system component will be apparent to those of ordinary skill in the art in view of the teachings herein.

PRU (70) delivers fluid to a patient via an infusion pump, such as a peristaltic infusion pump (84) (e.g., by B-Braun McGaw). Peristaltic infusion pump (84) is integrally attached to PRU (70), and uses peristaltic fingers to create a wavelike motion to induce fluid flow inside a flexible tube connected to a fluid reservoir. A drug cassette (86) is a generally rectangular shaped structure that is placed adjacent to peristaltic infusion pump (84). Drug cassette (86) of this example is made of a rigid thermoplastic such as, for example, polycarbonate. Drug cassette (86) has an internal cavity that houses IV tubing (22) made of a flexible thermoplastic such as, for example, polypropylene (e.g., Kelcourt). Drug cassette (86) receives tubing (22) via a port (88) and accurately and reliably positions exposed IV tubing (22) in contact with the peristaltic fingers of peristaltic infusion pump (84). IV tube set (22) attaches to a fluid vial (90), and a portion of the length of IV tube set (22) is contained within drug cassette (86). Another portion of IV tube set (22) lies external to drug cassette (86) to facilitate the interaction with peristaltic pump (84). IV tubing (22) is coiled within drug cassette (86) and has a length to reach a patient removed from the PRU (70). A fluid detection sensor (not shown) may be mounted to an inner wall of drug cassette (86). Such a fluid detection sensor may comprise any one of known fluid sensors, such as the MTI-2000 Fotonic Sensor, or the Microtrak-II CCD Laser Triangulation Sensor both by MTI Instruments Inc. IV tube set (22) may run through the fluid detection sensor before exiting drug cassette (86). PRU (70) may include features operable to prime IV tubing (22) with relative ease for a user. Various examples of how such priming may be provided are disclosed in U.S. Pat. No. 7,833,213, the disclosure of which is incorporated by reference herein.

In the present example, drug cassette (86) includes just one vial (90). However, it should be understood that some versions of drug cassette (86) may include several vials (90). Such vials (90) may include the same drug. Alternatively, a plurality of vials (90) associated with a single drug cassette (86) may include a variety of different kinds of drugs. In other words, a single drug cassette (86) may be used to selectively deliver two or more drugs simultaneously and/or in a particular sequence. While vials (90) are used in the present example, it should be understood that any other suitable type of container may be used as will be understood by those of ordinary skill in the art in view of the teachings herein. It should also be understood that some versions of PRU (70) may be configured to receive two or more drug cassettes (86). Each such drug cassette (86) may be associated with a single drug (e.g., different drug cassettes (86) used for different drugs), or each drug cassette (86) may be associated with a combination of drugs (e.g., different drug cassettes (86) used for different combinations of drugs).

FIG. 4 shows how components of system (10) interface with each other and with a patient. While not shown in FIG. 3, FIG. 4 shows how PRU (70) includes an integral ECG module (92) and integral cannula module (94). ECG module (92) is coupled with ECG (48) via ECG leads (68) extending from pass-through connection (24). Cannula module (94) is coupled with oral/nasal cannula (46), also through pass-through connection (24). Like modules (54, 58, 64, 66) described above, modules (92, 94) may be easily replaceable with other monitoring modules in the event of malfunction or technological advancement. Modules (92, 94) may also include all of the necessary hardware to operate their respective peripherals, and may be further coupled with a microprocessor-based electronic controller or computer located within PRU (70) and/or BMU (40).

As also shown in FIG. 4, PRU (70) of the present example is coupled with an external oxygen source (100), an external power source (102), and an external monitor (104). External oxygen source (100) may by regulated by one or more components of PRU (70), which may deliver oxygen from oxygen source (100) to the patient based on one or more parameters sensed by BMU (40), based on drug delivery from cassette (86), and/or based on other factors. External power source (102) may be used as a primary source of power for PRU (70), with a battery (96) being used as a backup power source. Alternatively, battery (96) may be used as a primary source of power for PRU, with external power source (102) being used for backup power and/or to charge battery (96). External monitor (104) may be used to supplement or to substitute the display features of touch screen assembly (42) and/or touch screen assembly (72). For instance, external monitor (104) may display information including patient physiological parameters, status of operation of system (10), warning alerts, etc. PRU (70) and/or BMU (40) may communicate with external monitor (104) via cable, wirelessly (e.g., via RF transmission, etc.), or otherwise. Other examples of components, features, and functionality that may be incorporated into PRU (70) will be described in greater detail below; while still further examples of components, features, and functionality that may be incorporated into PRU (70) will be apparent to those of ordinary skill in the art in view of the teachings herein.

II. Exemplary Oral-Nasal Cannula and Capnometer System

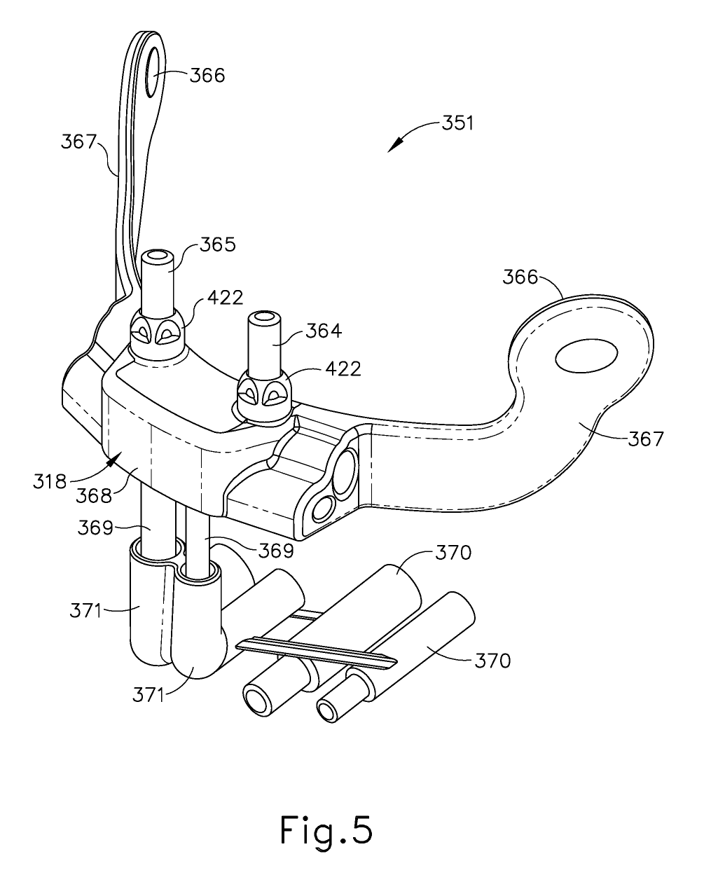

FIG. 5 shows an exemplary oral-nasal cannula (351) that may be used to provide oral-nasal cannula (46) of system (10) described above. In the present example, oral-nasal cannula (351) is made of soft and flexible material, such as polyurethane, silicon or some other elastomer; and is generally constructed by either injection-molding or liquid injection molding techniques. Oral-nasal cannula (351) includes a cannula cap (368) that is generally hollow and provides platform for supporting other features. The profile of oral-nasal cannula (351) is designed to easily adapt to patient anatomy. Oral-nasal cannula (351) includes adhesive pads (366) located on the patient side of cannula wings (367). Adhesive pads (366) are configured to adhere to and secure comfortably oral-nasal cannula (351) in place on the patient's face. Cannula cap (368) includes nasal prong (422) and oral prong (369). Cannula body (318) includes nasal prongs (364, 365) and oral prongs (370, 371). Nasal oxygen prongs (422) are mounted over prongs (364, 365). Prongs (422) have a tapered shape and fit co-axial around nasal prongs (364, 365). Prongs (422) include a number of holes to permit oxygen passage from within cannula cap (368) to the vicinity of the patient's nostrils.

In some instances, a conventional capnometer system incorporating a conventional oral-nasal cannula (351) may be relatively inefficient at accurately capturing carbon dioxide concentrations in gases exhaled by a patient. This may be due to the fact that some patients may exhale through their mouth versus through their nose at a different proportion than other patients. To further complicate matters, some patients may exhale through their mouth versus through their nose at one proportion at one part of a medical procedure; and then at another proportion at another part of the medical procedure. When an oral-nasal cannula (351) provides a shared oral and nasal exhalation output to a capnometer, this sharing of the exhalation path may provide a communication inefficiency, which may compromise the accuracy of carbon dioxide concentration readings by the capnometer. To the extent that the accuracy may be increased by providing a dedicated oral exhalation path with a dedicated oral exhalation capnometer, and a dedicated nasal exhalation path with a dedicated nasal exhalation capnometer, this kind of arrangement may significantly increase the cost and bulk of a capnometer system. It may therefore be desirable to provide a capnometer system that provides greater efficiency and/or accuracy over conventional capnometer systems, without providing unnecessary duplication of parts or unnecessary increases in expense.

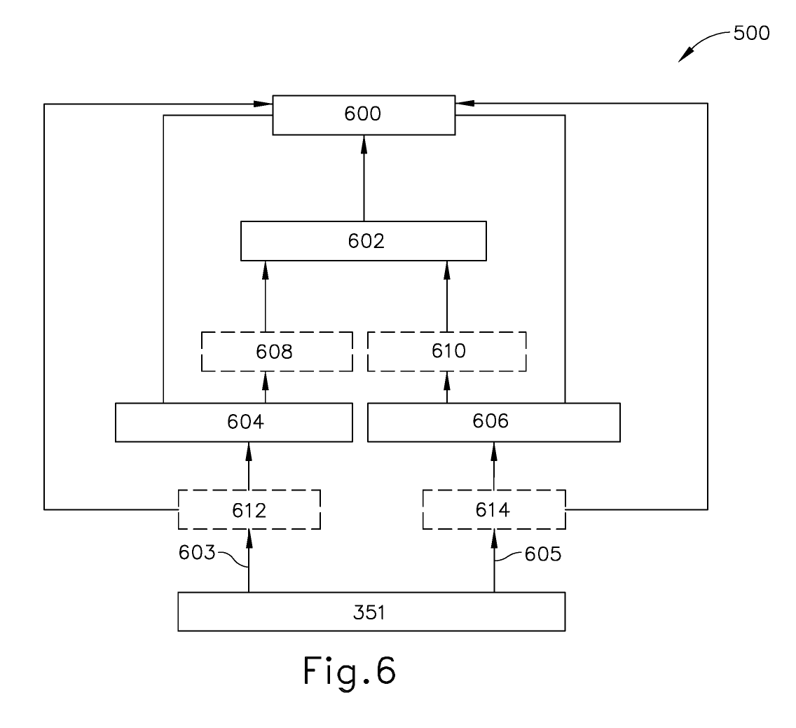

FIG. 6 shows an exemplary capnometer system (500) that incorporates oral-nasal cannula (351) and that may provide greater efficiency and/or accuracy over conventional capnometer systems. It should also be understood that capnometer system (500) may be readily integrated into system (10) described above. Various suitable ways in which capnometer system (500) may be integrated into system (10) will be apparent to those of ordinary skill in the art in view of the teachings herein. It should also be understood that capnometer system (500) may be used in a variety of other contexts, including as a standalone assembly or in combination with other components, without necessarily being integrated into system (10).

In the example shown in FIG. 6, oral-nasal cannula (351) has an oral gas pathway (603) connecting the output of one or more oral prongs (370, 371) to a capnometer (602); and a nasal gas pathway (605) connecting the output of one or more nasal prongs (364, 365) to the same capnometer (602). The nasal gas pathway (605) may thus communicate gases exhaled from one or both nasal openings of the patient. Oral and nasal gas pathways (603, 605) are fluidly isolated from each other en route to capnometer (602). Oral gas pathway (603) has an oral variable valve (604) that is controlled by a valve controller (600). Oral variable valve (604) has a variable orifice, such that valve (604) is adjustable to allow a variable flow of gas from oral-nasal cannula (351) to capnometer (602). Nasal gas pathway (605) has a nasal variable valve (606) that is also controlled by valve controller (600). Nasal variable valve (606) has a variable orifice, such that valve (606) is adjustable to allow a variable flow of gas from oral-nasal cannula (351) to capnometer (602).

It may be beneficial to position variable valves (604, 606) as close as possible to the patient's nose and mouth (e.g., in order to minimize lag in effect). In some versions, variable valves (604, 606) are incorporated directly into oral-nasal cannula (351). In some other versions, variable valves (604, 606) are mounted to a bed upon which the patient is supported. In some other versions, variable valves (604, 606) are incorporated into headphones that are worn by the patient. Other suitable locations for variable valves (604, 606) will be apparent to those of ordinary skill in the art in view of the teachings herein.

In the present example, the same valve controller (600) can adjust oral variable valve (604) and nasal variable valve (606) individually to allow varying amounts of gas from each into capnometer (602). Capnometer (602) may itself have a mixing chamber where gasses coming from oral variable valve (604) and nasal variable valve (606) may intermingle before being measured by capnometer (602) for exhaled carbon dioxide levels. Capnometer (602) is in communication with valve controller (600) so that characteristics measured by capnometer (602), such as carbon dioxide levels and respiratory rate, may be reported to the valve controller (600) in real time. Valve controller (600) itself may have a processor and memory, and may be configured with software for receiving data from and transmitting data to devices in communication with valve controller (600), such as the sensors (602, 612, 614) and valves (604, 606) described herein or other similar components. In some versions, valve controller (600) may be a standalone piece of equipment with connections for gases and data from the described components; while in other versions, valve controller (600) may be integrated into another device, such as capnometer (602), a BMU (40), a PRU (70), or some other piece of equipment. By way of example only, valve controller (600) may include a proportional-integral-derivative (PID) controller. Various suitable components and configurations that may be incorporated into valve controller (600) will be apparent to those of ordinary skill in the art in view of the teachings herein.

In some versions, oral gas pathway (603) may have an oral flow sensor (612) between oral-nasal cannula (351) and capnometer (602). Such an oral flow sensor (612) may provide data to valve controller (600) on the flow of gasses exhaled orally through oral-nasal cannula (351). Similarly, nasal gas pathway (605) may have a nasal flow sensor (614) between oral-nasal cannula (351) and capnometer (602). Such a nasal flow sensor (614) may provide data to valve controller (600) on the flow of gasses exhaled nasally through oral-nasal cannula (351). In this configuration, oral flow sensor (612) and nasal flow sensor (614) may provide information to valve controller (600) representing the ratio of how much the patient is exhaling orally versus how much the patient is exhaling nasally, so that valve controller (600) may adjust variable valves (604, 606) accordingly in response to the flow of gases. For example, if oral flow sensor (612) indicates that no exhaled gases are passing through oral gas pathway (603), valve controller (600) may cause oral variable valve (604) to close entirely in order to prevent backflow from capnometer (602), thereby maximizing the capnometer (602) receipt of nasally exhaled gasses passing through nasal variable valve (604). Still further examples may include, in addition to or in the alternative to flow sensors (612, 614), an oral check valve (608) and a nasal check valve (610) to prevent backflow of gasses from capnometer (602).

The particular components present in an oral/nasal gas pathway (603, 605) for an oral-nasal cannula (351), and the particular arrangement and configuration of capnometer system (500), may vary depending upon the intended use and implementation in light of factors such as desired cost and need for accuracy, and thus the examples shown and described above may vary. Other suitable components and arrangements that may be used to form capnometer system (500) will be apparent to those of ordinary skill in the art in view of the teachings herein.

III. Exemplary Methods of Managing Variable Valves

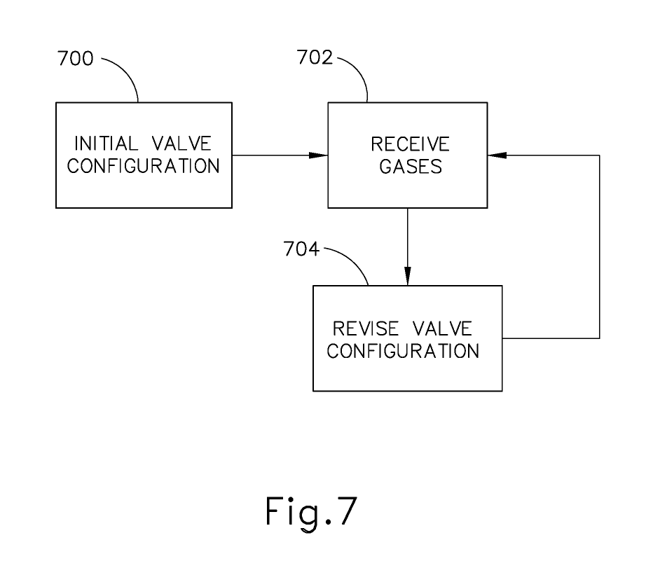

FIG. 7 shows an exemplary set of high level steps for managing a multi-input variable valve capnometer system such as capnometer system (500) described above. These management steps could be performed by an appropriately configured variable valve controller (600), BMU (40), PRU (70), and/or other device(s) that can receive, store, and execute a set of programmed instructions. For ease of discussion only, the following examples and descriptions will refer to a valve controller (600) of capnometer system (500) as the device that is configured to perform the steps.

At the beginning of the process, an initial valve configuration (block 700) is performed to adjust variable valves (604, 606) to an appropriate initial level. Exemplary methods of determining such appropriate initial levels will be described in greater detail below. Once the capnometer system (500) is configured and oral-nasal cannula (351) is placed on a patient, exhaled gases are received (block 702) by oral-nasal cannula (351) and travel along oral and nasal pathways (603, 605). As received gases are analyzed by sensors in capnometer (602), information garnered from the analysis may be used by valve controller (600) to revise and adjust (block 704) variable valves (604, 606) in order to change the configuration of oral and nasal pathways (603, 605) for subsequently received gases (block 702).

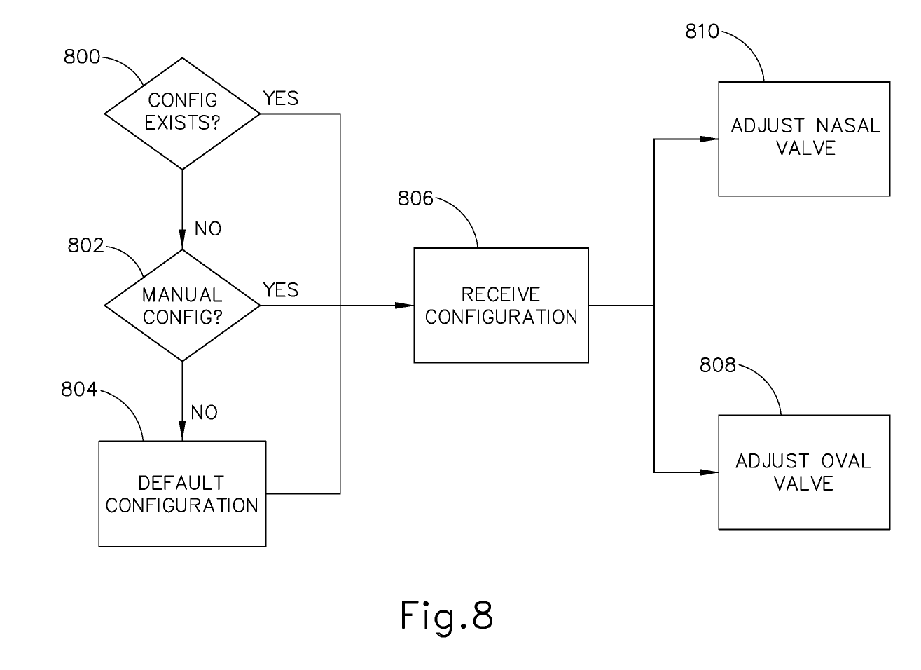

FIG. 8 shows an exemplary set of steps that may be performed to initially configure (block 700) variable valves (604, 606). Valve controller (600) may determine that a valve configuration profile exists (block 800) and is available from a remote source and/or a local source. A remote source may include (but is not limited to) a facility server or medical record server. A local source may include (but is not limited to) a memory, digital storage card, or storage device of valve controller (600), BMU (40), PRU (70) or another device in communication with valve controller (600). The remotely or locally available configuration may be patient or procedure specific, and may include configurations based upon past records or observations of a patient or procedure. For example, a particular patient may have been previously observed as breathing primarily through their nose. A patient specific configuration for that patient may initially configure the oral valve (604) at 10% open, and the nasal valve (606) at 100% open. Similarly, for a procedure where a patient is likely to breathe primarily through their mouth (e.g., when the patient has been previously observed as breathing primarily through their mouth), such as where medication or equipment used in the procedure affects a patient's conscious breathing patterns, a procedure specific configuration may initially configure the oral valve (604) at 90% open and the nasal valve (606) at 10% open.

If there is no remotely or locally available configuration (block 800), a manual configuration may be received from a user (block 802). The manual configuration may include a valve position for each valve, such as 0%-100% open, a numerical indicator such as 0-10, or a textual indication such as “mouth breather,” “nose breather,” or both, with each configuration value indicating a variable valve (604, 606) state for each of the variable valves (604, 606).

If no manual configuration is provided (block 802), a default configuration may be used (block 804). The default configuration could be a backup configuration present for each valve controller (600), and could indicate both variable valves (604, 606) being completely open, both variable valves (604, 606) being partially open, and any other valve configuration that a clinician might find useful. Once the configuration is located from a local or remote device (block 800), a manual configuration (block 802), or a default configuration (block 804), the configuration is received (block 806) from the source by valve controller (600). Once a configuration is available on valve controller (600), the configuration may be used to adjust (block 808) a nasal valve (606) and adjust (block 810) an oral valve (604) to an initial variable position.

Configurations may also include a variable valve (604, 606) adjustment routine. Such an adjustment routine may include defining a plurality of different valve states for each valve (604, 606) that may be stepped through to gradually change the flow through each valve (604, 606), a flow threshold for determining whether flow sensed by an oral flow sensor (612) or nasal flow sensor (614) indicates that gases are being exhaled along oral or nasal pathway (603, 605), and similar configurable characteristics.

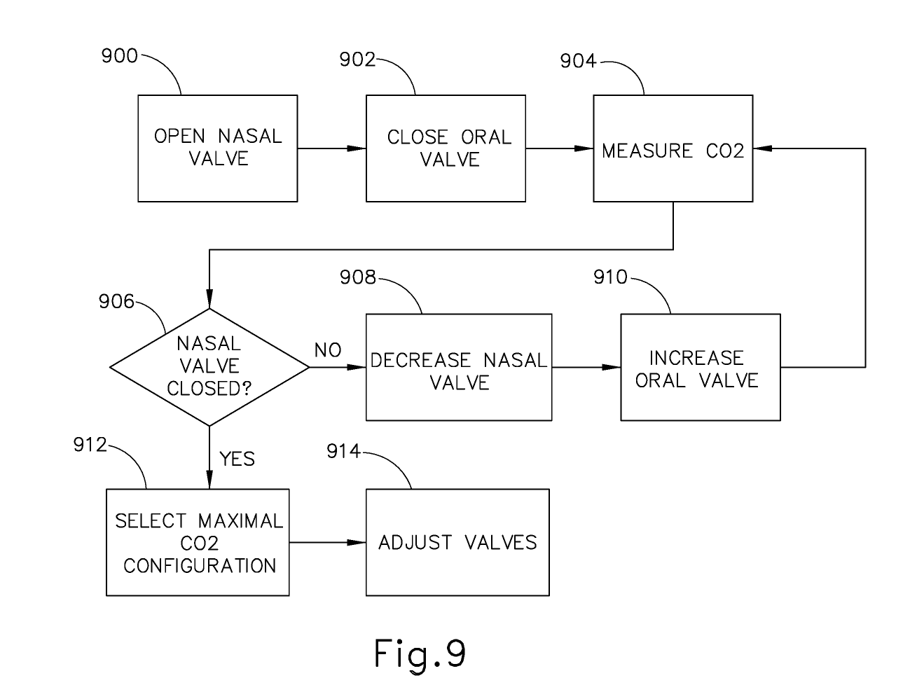

FIG. 9 shows an exemplary set of steps that may be performed to initially configure (block 700) a set of variable valves (604, 606) using a sweep method. This method of configuring valves (604, 606) could be performed in addition to or in the alternative to the steps described above in the context of FIG. 8. After placing oral-nasal cannula (351) on a patient, a nasal valve (606) may be opened fully (block 900) and an oral valve (604) may be closed fully (block 902). As the patient exhales gases through oral-nasal cannula (351), the exhaled gases will travel along oral and nasal pathways (603, 605), as restricted by valves (604, 606), and will be sensed by the capnometer (602) so that carbon dioxide levels within the exhaled gases can be measured (block 904).

Each carbon dioxide level measured (block 904) may be preserved during this process, or a set of maximum and minimum levels may be preserved. After carbon dioxide is measured (block 904), if nasal valve (906) is not fully closed (block 906), the valve opening of nasal valve (606) will be decreased (block 908) by some portion. The decrease may be 1%, 5%, 10%, or any similar percentage if valve (606) is controlled along a percentage spectrum; or may be any step along a set of configured valve steps or other valve opening measurements. Oral valve (604) opening may, in parallel or in sequence, be increased (block 910) at a same or similar proportion as nasal valve (606) was opened, including by a percentage, a configured step, or whatever other measurable difference between valve openings that system (500) may be configured to use.

After each valve opening has been decreased (block 908) and/or increased (block (910) respectively, a patient's exhaled gases will again be measured for carbon dioxide levels (block 904) with the measured data being preserved, either completely or as a maximum or minimum value. These steps (blocks 904, 906, 908, 910) may repeat until a maximum carbon dioxide output valve configuration is identified, until nasal valve (606) is fully closed, until oral valve (604) is fully open, until carbon dioxide levels decrease consistently enough to indicate that a maximum carbon dioxide has likely been identified, or until another similar point that indicates a valve configuration that results in maximum carbon dioxide intake to the capnometer (602) has been reached.

Operating in this manner, valve controller (600) and valves (604, 606) will sweep through a variety of valve configurations in an attempt to identify a configuration that will result in the maximum carbon dioxide intake to the capnometer (602). For example, if a patient breathes primarily through their nose, the maximal valve configuration identified by this process will likely be heavily weighted towards nasal valve (606) being partially or fully open and oral valve (604) being partially or fully closed. System (500) will operate similarly for primarily oral breathers, and mixed breathers, with valve openings varying in between. Additionally, the process could begin with an oral valve (604) completely open and a nasal valve (606) completely closed, or each valve (604, 606) open proportionally, or similar configurations, so long as the entire process sweeps and tests carbon dioxide at a variety of configurations. Once the looping steps end, a maximal carbon dioxide may be identified and selected (block 912) and valves (604, 606) may be adjusted (block 914) to the valve opening configuration associated with the maximal carbon dioxide and may thereafter be maintain at this selected valve opening configuration.

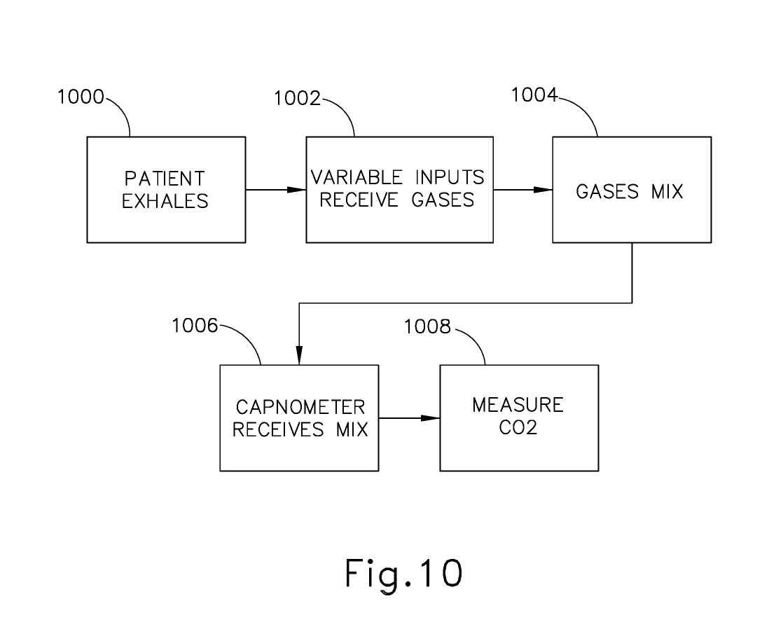

FIG. 10 shows an exemplary set of steps that may be performed to receive exhaled gases (block 702) via adjusted variable valves (604, 606) (e.g., with the states of valves (604, 606) being initially adjusted in accordance with the methods described above with reference to FIGS. 8-9). Once system (500) has been configured and oral-nasal cannula (351) has been fitted to a patient, as the patient exhales (block 1000), the exhaled gases will pass through (block 1002) the initially adjusted variable valves (604, 606). The received gases will mix (block 1004) as they arrive and are received (block 1006) by capnometer (602). As each stream of exhaled gases arrives, or based upon a configured or maximum sensor operation cycle, capnometer (602) will measure carbon dioxide levels (block 1008) in the exhaled gases, with valve controller (600) receiving the measurements from capnometer (602).

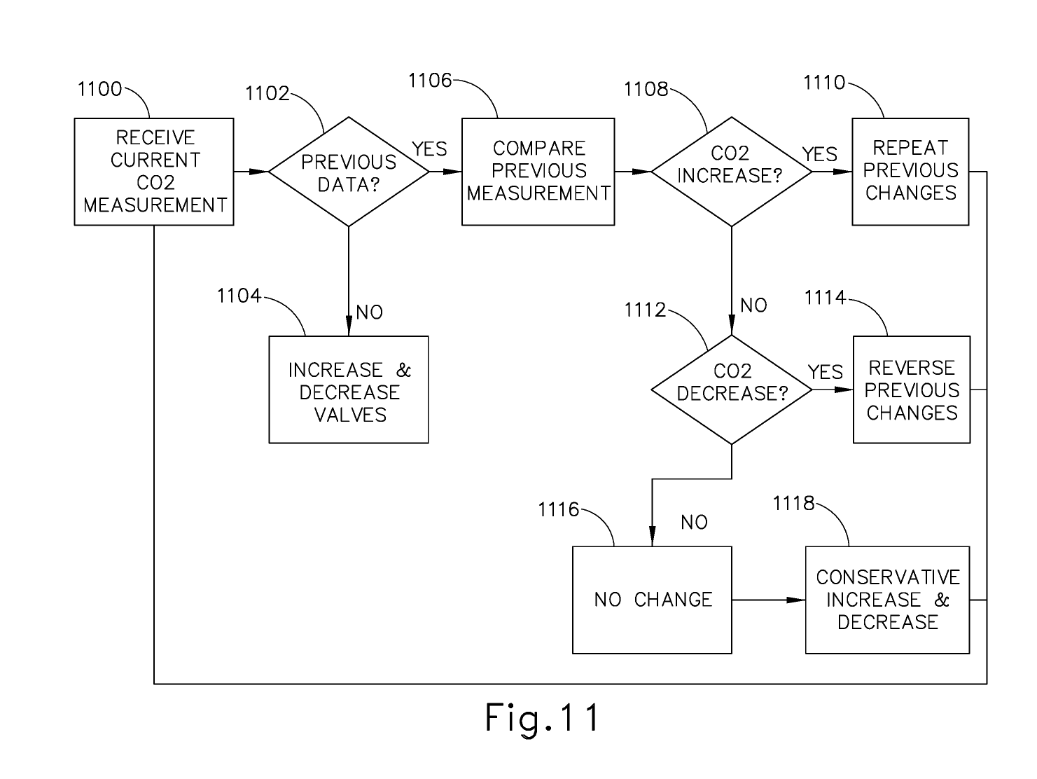

FIG. 11 shows an exemplary set of steps that may be performed to adjust (block 704) the states of variable valves (604, 606) in response to a set of sensor data. As data from capnometer (602) is received (block 1100) by valve controller (600), it is determined whether there is any historic data to compare the newly received data to (block 1102). If there is no historic data, such as when the process first begins, valve controller (600) may make an initial arbitrary valve adjustment (block 1104). For example, valve controller (600) may increase the opening of oral valve (604) and decrease the opening of nasal valve (606), or the opposite. This arbitrary change in the absence of any historic data for comparison establishes a baseline, generating a point for comparison moving forward.

Once historic data is available indicating carbon dioxide levels in the exhaled gases, the most recently received (block 1100) measurement may be compared against one or more previous measurements (block 1106). If the comparison (block 1106) indicates an increase in measured carbon dioxide as compared to the previous measurement(s), the most recent change in valve configuration may be repeated (block 1110). If the comparison (block 112) indicates a decrease in measured carbon dioxide as compared to the previous measurement(s), the most recent change in valve configuration may be reversed. When no significant change in carbon dioxide is detected in a current measurement (block 116), valve controller (600) may make a conservative arbitrary change (block 1118) in the openings of valve (604, 606). A conservative arbitrary change in valve configurations prevents the comparison data from stagnating and getting locked into an apparently maximized valve configuration when a more optimal valve configuration may exist. As each current carbon dioxide measurement is received (block 1100) and then processed and acted upon (blocks 1102-1118), the entire set of steps may be looped as a new current measurement is received (block 1100).

As an example of how the steps of FIG. 11 may be executed in practice, a patient may be fitted with an oral-nasal cannula (351) to capture their exhaled gases. Valve controller (600) may initially configure oral valve (604) and nasal valve (606) to each be at a 50% flow. As the patient exhales, the exhaled gases are captured and passed through oral and nasal gas pathways (603, 605) and limited by oral valve (604) and nasal valve (606), each at 50% flow. Some portion of the exhaled gases are received by capnometer (602) and the carbon dioxide levels are detected and received (block 1100) by valve controller (600). As an example, valve controller (600) may receive (block 1100) a set of data indicating that the carbon dioxide partial pressure (PCO₂) is 35 mmHg based upon the most recently captured data. Since this is the first data captured (block 1102), an arbitrary valve adjustment occurs (block 1104) resulting in nasal valve (606) being opened to 60% flow and oral valve (604) being closed to 40%.

A second set of data is received (block 1100) indicating that the patient's PCO₂ is now 37 mmHg. The second set of data is compared to the first set of data (block 1106), and it is determined that PCO₂ has measurably increased (block 1108) from the previous data set to the current data set. As a result, valve controller (600) will repeat the previous incremental change (block 1100), resulting in a valve configuration of 70% nasal flow and 30% oral flow. Following the same process, a third data set indicating PCO₂ of 39 mmHg may result in a valve configuration of 80% nasal flow and 20% oral flow, and a fourth data set indicating PCO2 of 42 mmHg may result in a valve configuration of 90% nasal flow and 10% oral flow.

If a fifth data set indicates PCO₂ of 39 mmHg, this will be determined to be a CO₂ decrease (block 1112) and will result in the previous change being fully or partially reversed (block 1114), resulting in a nasal flow of 85% and an oral flow of 15%. A sixth data set may indicate PCO₂ of 42 mmHg, which may be detected as an insignificant change (block 1116) from the previous or maximum PCO2 of 39-42 mmHg, resulting in a number of subsequent conservative valve state changes (block 1118), with subsequent readings indicating approximately 42 mmHg. The configuration of valves (604, 606) may be maintained at 85% nasal and 15% oral for the remainder of the procedure, or until a patient's breathing patterns change such that a significant change in measured PCO₂ results in further adjustment of the configuration or state of valves (604, 606). In this way, valve controller (600) will, in real time, adjust valves (604, 606) to maximize detected PCO₂ and adjust for varying and dynamic breathings patterns of the patient. While the examples above have generally described the combined valve opening percentage to be near 100%, this is an arbitrary total percentage and valve openings could be combined to exceed or fall short of a 100% total, such as where each valve (604, 606) is fully open.

In versions having an oral flow sensor (612) or nasal flow sensor (614), the sensor data indicating air flow through the passage in the direction of capnometer (602) may be used in the alternative to or in combination with the steps above. For example, if oral flow sensor (612) indicates no oral gas flow toward capnometer (602), valve controller (600) may cause the oral variable valve (604) to fully close, and nasal variable valve (606) to fully open. This may override any of the steps described above for a set period of time or until the flow sensor senses an increase in flow through oral gas pathway (603). Alternatively, flow sensors (612, 614) may be used merely as a single factor in determining the optimum configuration or state of valves (604, 606). For example, if gas flow is detected in a certain pathway (603, 605) by flow sensor (612, 614), the corresponding variable valve (604, 606) may be adjusted and opened by a step, stage, or set percentage from where it already is, so long as the flow is present.

The steps of FIG. 7-11 may be performed in simultaneously or in sequence, and singularly or in parallel, unless otherwise implicitly required or explicitly indicated by the descriptions. For example, in FIG. 7, a valve controller (600) may be both receiving gases (block 702) and revising valve configurations (block 704) simultaneously, and may, for example, be revising several valve configurations (block 704) based upon several sets of data in parallel. The nature of the processing of data and the sequences used may vary from version to version and may depend upon such factors as the particular hardware present in a valve controller (600) and a balance of speed, efficiency, and complexity of the resulting system.

IV. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus comprising: (a) an oral gas pathway configured to communicate gases exhaled orally by a patient; (b) a nasal gas pathway configured to communicate gases exhaled nasally through one or two nostrils of a patient; (c) an oral variable valve situated within the oral gas pathway, wherein the oral variable valve has an adjustable orifice that is configured to selectively restrict communication of gases exhaled orally through the oral gas pathway; (d) a nasal variable valve situated within the nasal gas pathway, wherein the nasal variable valve has an adjustable orifice that is configured to selectively restrict communication of gases exhaled nasally through the nasal gas pathway; (e) a capnometer, wherein the capnometer has a sensor configured to detect carbon dioxide in exhaled gas, wherein the capnometer is in fluid communication with the oral and nasal gas pathways such that the capnometer is configured to receive gases exhaled orally and nasally by a patient as restricted by the oral and nasal variable valves, respectively; and (f) a valve controller configured to receive data from the sensor of the capnometer, wherein the valve controller operable to adjust the oral variable valve and the nasal variable valve based on data from the sensor of the capnometer.

### Example 2

The apparatus of Example 1, further comprising: (a) an oral check valve situated within the oral gas pathway, wherein the oral check valve is configured to prevent backflow of gases through the oral gas input; and (b) a nasal check valve situated within the nasal gas pathway, wherein the nasal check valve is configured to prevent backflow of gases through the nasal gas input.

### Example 3

The apparatus of any one or more of Examples 1 through 2, further comprising:

(a) an oral flow sensor situated within the oral gas pathway and configured to sense the flow of gases through the oral gas pathway, wherein the valve controller is further configured to receive a set of oral flow data from the oral flow sensor and adjust the oral variable valve based upon the set of oral flow data; and (b) a nasal flow sensor situated within the nasal gas pathway and configured to sense the flow of gases through the nasal gas pathway, wherein the valve controller is further configured to receive a set of nasal flow data from the nasal flow sensor and adjust the nasal variable valve based upon the set of nasal flow data.

### Example 4

The apparatus of any one or more of Examples 1 through 3, further comprising an oral-nasal cannula, wherein the oral-nasal cannula is coupled with the oral gas pathway and the nasal gas pathway.

### Example 5

The apparatus of any one or more of Examples 1 through 4, wherein the oral gas pathway comprises a first set of one or more tubes, and wherein the nasal gas pathway comprises a second set of one or more tubes.

### Example 6

The apparatus of any one or more of Examples 1 through 5, wherein the valve controller is configured to adjust the nasal variable valve and the oral variable valve to an initial configuration based upon one or more of: (i) a patient specific valve configuration, (ii) a procedure specific valve configuration, (iii) a valve configuration defined by a clinician at the time of a procedure, and (iv) a default configuration.

### Example 7

The apparatus of any one or more of Examples 1 through 6, wherein the valve controller is configured to: (i) selectively adjust the oral variable valve to provide a first initial valve state providing a first initial degree of restriction through the oral gas pathway, and (ii) selectively adjust the nasal variable valve to provide a second initial valve state providing a second initial degree of restriction through the nasal gas pathway.

### Example 8

The apparatus of Example 7, wherein the valve controller is further configured to: (i) receive a first set of carbon dioxide data from the capnometer, (ii) adjust the oral variable valve to an first arbitrary adjusted valve state, wherein the first arbitrary adjusted valve state provides a different degree of restriction than the first initial degree of restriction, (iii) receive a second set of carbon dioxide data from the capnometer, (iv) compare the first set of carbon dioxide data to the second set of carbon dioxide data to create a carbon dioxide comparison, and (v) identify an optimized oral valve state based upon the carbon dioxide comparison.

### Example 9

The apparatus of Example 8, wherein the valve controller is configured to identify the optimized oral valve state by: (i) selecting a valve state that allows a greater flow than the first arbitrary adjusted valve state when the carbon dioxide comparison indicates the first set of carbon dioxide data has a higher concentration of carbon dioxide than the second set of carbon dioxide data, and (ii) selecting a valve position that allows a lesser flow than the first arbitrary adjusted valve state when the carbon dioxide comparison indicates the first set of carbon dioxide data has a lower concentration of carbon dioxide than the second set of carbon dioxide data.

### Example 10

The apparatus of any one or more of Examples 8 through 9, wherein the valve controller is further configured to adjust the oral variable valve to the optimized position a plurality of times in real time during a procedure.

### Example 11

The apparatus of any one or more of Examples 8 through 10, wherein the valve controller is further configured to: (i) adjust the nasal variable valve to a second arbitrary adjusted valve state, wherein the second arbitrary adjusted valve state provides a different degree of restriction than the second initial degree of restriction, and (ii) identify an optimized nasal valve state based upon the carbon dioxide comparison.

### Example 12

The apparatus of Example 11, wherein the valve controller is configured to identify the optimized oral valve state by: (i) selecting an oral valve state that allows a greater flow than the first arbitrary adjusted valve state when the carbon dioxide comparison indicates the first set of carbon dioxide data has a higher concentration of carbon dioxide than the second set of carbon dioxide data, and (ii) selecting an oral valve position that allows a lesser flow than the first arbitrary adjusted valve state when the carbon dioxide comparison indicates the first set of carbon dioxide data has a lower concentration of carbon dioxide than the second set of carbon dioxide data; wherein the valve controller is configured to identify the optimized nasal valve state by: (i) selecting a nasal valve state that allows a greater flow than the second arbitrary adjusted valve state when the carbon dioxide comparison indicates the first set of carbon dioxide data has a higher concentration of carbon dioxide than the second set of carbon dioxide data, and (ii) selecting a nasal valve position that allows a lesser flow than the second arbitrary adjusted valve state when the carbon dioxide comparison indicates the first set of carbon dioxide data has a lower concentration of carbon dioxide than the second set of carbon dioxide data.

### Example 13

The apparatus of any one or more of Examples 1 through 12, wherein the valve controller is further configured to determine an initial oral variable valve state and an initial nasal variable valve state by: (i) adjusting one of the oral variable valve or the nasal variable valve to a fully open position, (ii) adjusting the other of the oral variable valve or the nasal variable valve to a fully closed position, and (iii) repeating, until a maximal carbon dioxide concentration is identified: (A) receiving a first set of test data from the capnometer, (B) decreasing the flow of the more open variable valve by a volume, (C) increasing the flow of the more closed variable valve by a volume, (D) receiving a second set of test data from the capnometer, (E) determining which of the first set of test data or the second set of test data indicates a highest carbon dioxide concentration, and (F) saving the highest carbon dioxide concentration as a potential maximal carbon dioxide concentration, and associating the current flow of the oral variable valve and the current flow of the nasal variable valve with the potential maximal carbon dioxide concentration, and (iv) after the maximal carbon dioxide concentration is identified: (A) setting the initial oral variable valve state as the oral variable valve state used when the maximal carbon dioxide concentration was identified, and (B) setting the initial nasal variable valve state as the nasal variable valve state used when the maximal carbon dioxide concentration was identified.

### Example 14

The apparatus of any one or more of Examples 1 through 13, wherein the valve controller comprises a proportional-integral-derivative (PID) controller.

### Example 15

The apparatus of any one or more of Examples 1 through 14, wherein the valve controller and the capnometer are integrated with a bedside monitor unit, wherein the bedside monitor unit is configured to monitor a plurality of biological parameters of a patient.

### Example 16

The apparatus of Example 15, wherein the bedside monitor unit is coupled with an automated drug delivery unit, wherein the automated drug delivery unit is configured to provide automated drug delivery to a patient based at least in part on real time data from the capnometer.

### Example 17

An apparatus comprising: (a) an oral variable valve having an adjustable orifice that is configured to selectively restrict communication of gases exhaled orally through an oral gas pathway; (b) a nasal variable valve having an adjustable orifice that is configured to selectively restrict communication of gases exhaled nasally through a nasal gas pathway; (c) a capnometer, wherein the capnometer has a sensor configured to detect carbon dioxide in exhaled gas, wherein the capnometer is in fluid communication with the oral and nasal gas pathways such that the capnometer is configured to receive gases exhaled orally and nasally by a patient as restricted by the oral and nasal variable valves, respectively; and (d) a valve controller configured to receive data from the sensor of the capnometer, wherein the valve controller is operable to: (i) identify an optimized combination of oral and nasal variable valve states based on data from the sensor of the capnometer, and (ii) adjust the oral and nasal variable valves based on the identified optimized combination.

### Example 18

A method comprising the steps: (a) adjusting a nasal variable valve to an initial nasal valve state, wherein the nasal variable valve is configured to selectively restrict communication of gas through a nasal gas pathway; (b) adjusting an oral variable valve to an initial oral valve state, wherein the oral variable valve is configured to selectively restrict communication of gas through a oral gas pathway; (c) receiving, at a valve controller, a plurality of sets of carbon dioxide data generated by a capnometer, wherein the capnometer generates the carbon dioxide data from gases exhaled from a patient as received via the nasal gas pathway and the oral gas pathway; (d) determining, based upon the plurality of sets of carbon dioxide data, an optimal nasal valve state and an optimal oral valve state, wherein the optimal nasal valve state and the optimal oral valve state result in a maximal carbon dioxide concentration for a set of carbon dioxide data from the plurality of sets of carbon dioxide data; and (e) adjusting the nasal variable valve to the optimal nasal valve state and adjusting the oral variable valve to the optimal oral valve state.

### Example 19

The method of Example 18, wherein adjusting the nasal variable valve to the optimal nasal valve state and adjusting the oral variable valve to the optimal oral valve state occurs is repeated in real time in response to updated carbon dioxide data received during a procedure.

### Example 20

The method of any one or more of Examples 18 through 19, further comprising activating an automated drug delivery system to adjust dosage of a drug administered to a patient based on carbon dioxide data received from the capnometer after the act of adjusting the nasal variable valve to the optimal nasal valve state and adjusting the oral variable valve to the optimal oral valve state.

V. Miscellaneous

It should be understood that any of the examples described herein may include various other features in addition to or in lieu of those described above. By way of example only, any of the devices herein may also include one or more of the various features disclosed in any of the various references that are incorporated by reference herein. It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus comprising:
(a) an oral gas pathway configured to communicate gases exhaled orally by a patient;
(b) a nasal gas pathway configured to communicate gases exhaled nasally through one or two nostrils of a patient;
(c) an oral variable valve situated within the oral gas pathway, wherein the oral variable valve has an adjustable orifice that is configured to selectively restrict communication of gases exhaled orally through the oral gas pathway;
(d) a nasal variable valve situated within the nasal gas pathway, wherein the nasal variable valve has an adjustable orifice that is configured to selectively restrict communication of gases exhaled nasally through the nasal gas pathway;
(e) a capnometer, wherein the capnometer has a sensor configured to detect carbon dioxide in exhaled gas, wherein the capnometer is in fluid communication with the oral and nasal gas pathways such that the capnometer is configured to receive gases exhaled orally and nasally by a patient as restricted by the oral and nasal variable valves, respectively; and
(f) a valve controller configured to receive data from the sensor of the capnometer, wherein the valve controller operable to adjust the oral variable valve and the nasal variable valve based on data from the sensor of the capnometer.

2. The apparatus of claim 1, further comprising:
(a) an oral check valve situated within the oral gas pathway, wherein the oral check valve is configured to prevent backflow of gases through the oral gas input; and
(b) a nasal check valve situated within the nasal gas pathway, wherein the nasal check valve is configured to prevent backflow of gases through the nasal gas input.

3. The apparatus of claim 1, further comprising:
(a) an oral flow sensor situated within the oral gas pathway and configured to sense the flow of gases through the oral gas pathway, wherein the valve controller is further configured to receive a set of oral flow data from the oral flow sensor and adjust the oral variable valve based upon the set of oral flow data; and
(b) a nasal flow sensor situated within the nasal gas pathway and configured to sense the flow of gases through the nasal gas pathway, wherein the valve controller is further configured to receive a set of nasal flow data from the nasal flow sensor and adjust the nasal variable valve based upon the set of nasal flow data.

4. The apparatus of claim 1, further comprising an oral-nasal cannula, wherein the oral-nasal cannula is coupled with the oral gas pathway and the nasal gas pathway.

5. The apparatus of claim 1, wherein the oral gas pathway comprises a first set of one or more tubes, and wherein the nasal gas pathway comprises a second set of one or more tubes.

6. The apparatus of claim 1, wherein the valve controller is configured to adjust the nasal variable valve and the oral variable valve to an initial configuration based upon one or more of:
(i) a patient specific valve configuration,
(ii) a procedure specific valve configuration,
(iii) a valve configuration defined by a clinician at the time of a procedure, and
(iv) a default configuration.

7. The apparatus of claim 1, wherein the valve controller is configured to:
(i) selectively adjust the oral variable valve to provide a first initial valve state providing a first initial degree of restriction through the oral gas pathway, and
(ii) selectively adjust the nasal variable valve to provide a second initial valve state providing a second initial degree of restriction through the nasal gas pathway.

8. The apparatus of claim 7, wherein the valve controller is further configured to:
(i) receive a first set of carbon dioxide data from the capnometer,
(ii) adjust the oral variable valve to an first arbitrary adjusted valve state, wherein the first arbitrary adjusted valve state provides a different degree of restriction than the first initial degree of restriction,
(iii) receive a second set of carbon dioxide data from the capnometer,
(iv) compare the first set of carbon dioxide data to the second set of carbon dioxide data to create a carbon dioxide comparison, and
(v) identify an optimized oral valve state based upon the carbon dioxide comparison.

9. The apparatus of claim 8, wherein the valve controller is configured to identify the optimized oral valve state by:
(i) selecting a valve state that allows a greater flow than the first arbitrary adjusted valve state when the carbon dioxide comparison indicates the first set of carbon dioxide data has a higher concentration of carbon dioxide than the second set of carbon dioxide data, and
(ii) selecting a valve position that allows a lesser flow than the first arbitrary adjusted valve state when the carbon dioxide comparison indicates the first set of carbon dioxide data has a lower concentration of carbon dioxide than the second set of carbon dioxide data.

10. The apparatus of claim 8, wherein the valve controller is further configured to adjust the oral variable valve to the optimized position a plurality of times in real time during a procedure.

11. The apparatus of claim 8, wherein the valve controller is further configured to:
(i) adjust the nasal variable valve to a second arbitrary adjusted valve state, wherein the second arbitrary adjusted valve state provides a different degree of restriction than the second initial degree of restriction, and
(ii) identify an optimized nasal valve state based upon the carbon dioxide comparison.

12. The apparatus of claim 11, wherein the valve controller is configured to identify the optimized oral valve state by:
(i) selecting an oral valve state that allows a greater flow than the first arbitrary adjusted valve state when the carbon dioxide comparison indicates the first set of carbon dioxide data has a higher concentration of carbon dioxide than the second set of carbon dioxide data, and
(ii) selecting an oral valve position that allows a lesser flow than the first arbitrary adjusted valve state when the carbon dioxide comparison indicates the first set of carbon dioxide data has a lower concentration of carbon dioxide than the second set of carbon dioxide data;
wherein the valve controller is configured to identify the optimized nasal valve state by:
(i) selecting a nasal valve state that allows a greater flow than the second arbitrary adjusted valve state when the carbon dioxide comparison indicates the first set of carbon dioxide data has a higher concentration of carbon dioxide than the second set of carbon dioxide data, and
(ii) selecting a nasal valve position that allows a lesser flow than the second arbitrary adjusted valve state when the carbon dioxide comparison indicates the first set of carbon dioxide data has a lower concentration of carbon dioxide than the second set of carbon dioxide data.

13. The apparatus of claim 1, wherein the valve controller is further configured to determine an initial oral variable valve state and an initial nasal variable valve state by:
(i) adjusting one of the oral variable valve or the nasal variable valve to a fully open position,
(ii) adjusting the other of the oral variable valve or the nasal variable valve to a fully closed position, and
(iii) repeating, until a maximal carbon dioxide concentration is identified:
(A) receiving a first set of test data from the capnometer,
(B) decreasing the flow of the more open variable valve by a volume,
(C) increasing the flow of the more closed variable valve by a volume,
(D) receiving a second set of test data from the capnometer,
(E) determining which of the first set of test data or the second set of test data indicates a highest carbon dioxide concentration, and
(F) saving the highest carbon dioxide concentration as a potential maximal carbon dioxide concentration, and associating the current flow of the oral variable valve and the current flow of the nasal variable valve with the potential maximal carbon dioxide concentration, and
(iv) after the maximal carbon dioxide concentration is identified:
(A) setting the initial oral variable valve state as the oral variable valve state used when the maximal carbon dioxide concentration was identified, and
(B) setting the initial nasal variable valve state as the nasal variable valve state used when the maximal carbon dioxide concentration was identified.

14. The apparatus of claim 1, wherein the valve controller comprises a proportional-integral-derivative (PID) controller.

15. The apparatus of claim 1, wherein the valve controller and the capnometer are integrated with a bedside monitor unit, wherein the bedside monitor unit is configured to monitor a plurality of biological parameters of a patient.

16. The apparatus of claim 15, wherein the bedside monitor unit is coupled with an automated drug delivery unit, wherein the automated drug delivery unit is configured to provide automated drug delivery to a patient based at least in part on real time data from the capnometer.

17. An apparatus comprising:
(a) an oral variable valve having an adjustable orifice that is configured to selectively restrict communication of gases exhaled orally through an oral gas pathway;
(b) a nasal variable valve having an adjustable orifice that is configured to selectively restrict communication of gases exhaled nasally through a nasal gas pathway;
(c) a capnometer, wherein the capnometer has a sensor configured to detect carbon dioxide in exhaled gas, wherein the capnometer is in fluid communication with the oral and nasal gas pathways such that the capnometer is configured to receive gases exhaled orally and nasally by a patient as restricted by the oral and nasal variable valves, respectively; and
(d) a valve controller configured to receive data from the sensor of the capnometer, wherein the valve controller is operable to:
(i) identify an optimized combination of oral and nasal variable valve states based on data from the sensor of the capnometer, and
(ii) adjust the oral and nasal variable valves based on the identified optimized combination.

18. A method comprising the steps:
(a) adjusting a nasal variable valve to an initial nasal valve state, wherein the nasal variable valve is configured to selectively restrict communication of gas through a nasal gas pathway;
(b) adjusting an oral variable valve to an initial oral valve state, wherein the oral variable valve is configured to selectively restrict communication of gas through a oral gas pathway;
(c) receiving, at a valve controller, a plurality of sets of carbon dioxide data generated by a capnometer, wherein the capnometer generates the carbon dioxide data from gases exhaled from a patient as received via the nasal gas pathway and the oral gas pathway;
(d) determining, based upon the plurality of sets of carbon dioxide data, an optimal nasal valve state and an optimal oral valve state, wherein the optimal nasal valve state and the optimal oral valve state result in a maximal carbon dioxide concentration for a set of carbon dioxide data from the plurality of sets of carbon dioxide data; and
(e) adjusting the nasal variable valve to the optimal nasal valve state and adjusting the oral variable valve to the optimal oral valve state.

19. The method of claim 18, wherein adjusting the nasal variable valve to the optimal nasal valve state and adjusting the oral variable valve to the optimal oral valve state occurs is repeated in real time in response to updated carbon dioxide data received during a procedure.

20. The method of claim 18, further comprising activating an automated drug delivery system to adjust dosage of a drug administered to a patient based on carbon dioxide data received from the capnometer after the act of adjusting the nasal variable valve to the optimal nasal valve state and adjusting the oral variable valve to the optimal oral valve state.
